(19) United States
(12) Patent Application Publication
(10) Pub. No.: US 2026/0184666 A1
(43) Pub. Date: Jul. 2, 2026

(54) HYDROGENATION PROCESS AND REACTION SYSTEM FOR HYDROGENATION

(71) Applicant: BASF SE, Ludwigshafen am Rhein (DE)

(72) Inventors: Myung Un WON, Yeosu (KR); Man Gyun PARK, Yeosu (KR); Stefan ENGELS, Ludwigshafen am Rhein (DE); Matthias HINRICHS, Ludwigshafen am Rhein (DE); Steffen OEHLENSCHLAEGER, Ludwigshafen am Rhein (DE); Hans SCHUYTEN, Antwerp (BE)

(21) Appl. No.: 19/132,068

(22) PCT Filed: Nov. 23, 2023
(86) PCT No.: PCT/EP2023/082886

§ 371 Date: May 22, 2025

(30) Foreign Application Priority Data
Nov. 24, 2022 (WO) PCT/CN2022/134048

(51) Int. Cl. C07C 209/32; B01J 19/00

(52) U.S. Cl. CPC C07C 209/32; B01J 19/0013

(57) ABSTRACT

In a first aspect, the invention relates to a hydrogenation process for the conversion of a compound comprising reacting the compound in a liquid medium with hydrogen in the presence of a heterogeneous hydrogenation catalyst in a reaction vessel comprised in a reaction apparatus, the process comprising: (i) transferring at least a part of the thermal energy generated in the reaction vessel to a heat transfer medium in a heat exchanger HE, preferably to a heat transfer medium stream HTMS1, obtaining a heat transfer medium stream HTMS2, which has an increased thermal energy content compared to HTMS1; (ii) increasing the pressure of the heat transfer medium stream HTMS2 having a pressure p₂, thereby obtaining a heat transfer medium stream HTMS3, which has an increased pressure p₃ compared to HTMS2. A second aspect of the invention relates to a reaction system for hydrogenation.

In a first aspect, the invention relates to a hydrogenation process for the conversion of a compound comprising reacting the compound in a liquid medium with hydrogen in the presence of a heterogeneous hydrogenation catalyst in a reaction vessel comprised in a reaction apparatus, the process comprising: (i) transferring at least a part of the thermal energy generated in the reaction vessel to a heat transfer medium in a heat exchanger HE, preferably to a heat transfer medium stream HTMS1, obtaining a heat transfer medium stream HTMS2, which has an increased thermal energy content compared to HTMS1; (ii) increasing the pressure of the heat transfer medium stream HTMS2 having a pressure p₂, thereby obtaining a heat transfer medium stream HTMS3, which has an increased pressure p₃ compared to HTMS2. A second aspect of the invention relates to a reaction system for hydrogenation.

Hydrogenation is a type of reaction that is used in many fields of applications. The reaction is characterized by the use of hydrogen, which is used as a reducing agent for a substrate, typically with additional use of a catalyst. Common reactants used in a hydrogenation reaction are, for example, alkenes, alkynes, aldehydes, ketones, esters, carboxylic acids and compounds having nitro groups. From the later, dinitro toluene is often used for hydrogenation reactions since the respective reaction product toluene diamine is the precursor of toluene diisocyanate, which is in turn the relevant isocyanate monomer in the preparation of polyurethane.

In existing technology, toluene diamine is produced by hydrogenation of dinitro toluene, wherein the reaction is strongly exothermic and sets a considerable amount of thermal energy free. Said reaction heat has to be removed, otherwise the reaction could not be carried out in a controlled manner for a prolonged period of time. Common means for removing the reaction heat are cooling systems, which normally use water as cooling medium. In one or more cooling loops, the reaction heat is taken off and for example then the cooling medium is disposed, since the cooling water in the end is discharged into waters. Especially nowadays, there is an overall demand for energetically efficient systems, i.e. systems, which are capable of not wasting but rather using generated energy for a meaningful purpose. Since steam, especially water steam, is an efficient energy carrier, which is used in many industrial processes, it could be considered to transfer the reaction heat of a hydrogenation reaction to steam.

Reactor setups for hydrogenation of dinitro toluene are described in the art, for example, WO 00/30743 A1 discloses a reactor of cylindrical construction for the continuous performance of solid gas-liquid, liquid-liquid or gas-liquid reactions, having a downwardly directed jet nozzle arranged in the upper reactor region, via which the starting materials and the reaction mixture are fed, and having a draw-off preferably in the lower reactor region, via which the jet nozzle is fed. Further described is a continuous process for carrying out gas-liquid or gas liquid-solid reactions in said reactor.

WO 00/35852 A1 is related to a process for preparing amines by hydrogenation of nitro compounds, characterized in that the hydrogenation is carried out in a vertical reactor whose length is greater than diameter with a downwardly directed jet nozzle arranged in the upper region of the reactor, via which the feeding materials and the reaction mixture are fed, and with a draw-off at any point in the reactor, via which the reaction mixture is fed in an external circuit back to the reactor.

However, the reaction temperature in most of the existing reactor setups is not high enough to be used efficiently, for example, for generation of steam with a higher grade, which can be used economically.

Thus, the problem underlying the present invention was to provide a way for overcoming the lack of current technology, especially to provide a process and a reaction system for recovering reaction heat from hydrogenation more efficiently, for example, by providing steam with a sufficiently high grade.

### 1^{st} Aspect—Hydrogenation Process

In a first aspect, the invention is directed to a hydrogenation process for the conversion of a compound comprising reacting the compound in a liquid medium with hydrogen in the presence of a heterogeneous hydrogenation catalyst in a reaction vessel comprised in a reaction apparatus, the process comprising
(i) transferring at least a part of the thermal energy generated in the reaction vessel to a heat transfer medium in a heat exchanger HE, preferably to a heat transfer medium stream HTMS1, obtaining a heat transfer medium stream HTMS2, which has an increased thermal energy content compared to HTMS1;
(ii) increasing the pressure of the heat transfer medium stream HTMS2 having a pressure p₂, thereby obtaining a heat transfer medium stream HTMS3, which has an increased pressure p₃ compared to HTMS2.

In some preferred embodiments, the hydrogenation process is a process for the hydrogenation of a compound having at least one nitro group into the corresponding compound having at least one amino group comprising reacting the compound having at least one nitro group in a liquid medium with hydrogen in the presence of a heterogeneous hydrogenation catalyst in a reaction vessel.

As indicated in further detail below, the hydrogenation process allows a favorable energetic balance in that at least 25% of the thermal energy generated in the reaction vessel are transferred to the heat transfer medium.

A heat exchanger is a device that transfers thermal energy from one material stream to another. While direct exchangers are known to the skilled person, the heat exchangers in the context of the present invention are preferably heat exchangers, wherein the material streams are not in direct contact with each other but are rather spatially separated, so that there is only an exchange of thermal energy possible, i.e. there are at least two compartments, which are spatially separated but are in thermal contact. Heat transfer medium stream HTMS1, which enters the heat exchanger HE, has preferably a temperature T₁ in the range of from >0 to 140° C., more preferably in the range of from 20 to 140° C., more preferably in the range of from 90 to 120° C. HTMS1 has a pressure p₁, which is ≥p₂, wherein the value of p₁ and the absolute value of the difference between p₂ and p₁ depends on the reaction apparatus, the set-up and arrangement of the lines etc., which a person skilled in the art is familiar with.

In some preferred embodiments of the hydrogenation process, the pressure difference between p₃ and p₂ [Δp(p₃−p₂)] is in the range of from 1 to 40 bar.

In some preferred embodiments of the hydrogenation process, the heat transfer medium stream HTMS2 has a pressure p₂ in the range of from −0.9 to 3 barg, preferably in the range of from −0.7 barg to 1 barg. Heat transfer medium stream HTMS2 preferably has a temperature T₂, which is equal or greater than the temperature at the boiling point (T_{BP}) of the heat transfer medium at a pressure p₂.

In some preferred embodiments of the hydrogenation process, the heat transfer medium stream HTMS3 has a pressure p₃ in the range of from 0.1 to 40 barg, preferably in the range of from 3 barg to 20 barg.

In some preferred embodiments of the hydrogenation process, increasing the pressure of the heat transfer medium stream HTMS2 having a pressure p₂ in (ii) is done by mechanical compression, preferably in a mechanical compression unit MC. Especially if the heat transfer medium stream HTMS2 has a low pressure, for example, a pressure p₂ in the range of from −0.9 to 3 barg, it is advantageous to do a compression by an additional mechanical steam compressor to pressurize the steam to pressure p₃ (pressure of HTMS3) in the range of from p₂+1 barg to 40 barg, preferably to pressure p₃ in the range of from p₂+2 to 20 barg. Even if additional electrical power input is needed for the additional mechanical compression, the overall energetic balance is still favorable. Heat transfer medium stream HTMS3 preferably has a temperature T₃, which is ≥the temperature at the boiling point (T_{BP}) of the heat transfer medium at a pressure p₃+3 K.

In some preferred embodiments of the hydrogenation process, (ii) comprises:
(ii.1) increasing the temperature of heat transfer medium stream HTMS2 having a temperature T₂ to a temperature T₂ₐ, which is higher than T₂, preferably in an overheater OH, thereby obtaining a heat transfer medium stream HTMS2a having a pressure p₂ₐ and a temperature T₂ₐ;
(ii.2) compressing HTMS2a, thereby obtaining a heat transfer medium stream HTMS3a, which has an increased pressure p₃ₐ compared to HTMS2 and HTMS2a respectively and a temperature T₃ₐ.

An overheater is a device used or usable for increasing the temperature of a heat transfer medium stream, which is partially in gaseous state, to a temperature higher than the vaporization point of the heat transfer medium at the absolute pressure where the temperature is measured, without significant change in pressure, i.e. the pressure after the overheater of the heat transfer medium stream is still only slightly lower than p₂, preferably p₂ₐ is in the range of from p₂ to p₂−300 mbar, more preferably p₂ₐ is in the range of from p₂ to p₂−100 mbar. In case of the heat transfer medium being or mostly consisting of water, such a heat transfer medium having a pressure p₂ₐ and a temperature T₂ₐ is called superheated steam or overheated steam. Saturated steam has several disadvantages, for example, it contains small droplets of water which can mechanically damage the compressor and have to be removed when said saturated steam is put to a use. Superheated or overheated steam can cool from T₂ₐ down to a temperature at the boiling point at a given pressure T_{BP}+at least 3 K, i.e. lose internal energy, by some amount, resulting in a lowering of its temperature without changing state, i.e. (partially) condensing, from the gaseous state to a mixture of saturated vapor and liquid. The same applies when the pressure is increased by some amount—also then, no change of aggregate state takes place.

In some preferred embodiments of the hydrogenation process, T₂ₐ is in the range of from the temperature at the boiling point (T_{BP}) of the heat transfer medium at a pressure p₂ₐ+3 K to temperature at the boiling point of the heat transfer medium (T_{BP}) at a pressure p₂ₐ+40 K and/or T₃ₐ is the temperature at the boiling point (T_{BP}) of the heat transfer medium at a pressure p₃ₐ+3 K. Thus, preferably, T₂ₐ is in the range of from (T_{BP}+3 K) to (T_{BP}+40 K) at pressure p₂ₐ and/or T₃ₐ is ≥the temperature at the boiling point (T_{BP}) of the heat transfer medium at a pressure p₃ₐ+3 K.

In some preferred embodiments of the hydrogenation process, in (ii) or (ii.2), the compression, preferably the mechanical compression, more preferably the mechanical compression in a mechanical compression unit MC is done in one compression stage, wherein the pressure of the heat transfer medium stream HTMS2 or HTMS2a respectively is increased from p₁ to p₂. Furthermore, in some embodiments with one compression stage, liquid heat transfer medium can be fed to the heat transfer medium stream HTMS2 or HTMS2a before the compression step, in order to avoid a heating-up of the heat transfer medium step due to the compression to a temperature above the design temperature of the compressor. The design temperature of a compressor is the temperature for which the compressor is engineered and built by the manufacturer in view of materials used, dimensions etc. so that the compressor is suitable to work at this temperature. Design temperature is normally determined based on the maximum normal operating temperature for which the compressor is intended to be used at.

In some preferred embodiments of the hydrogenation process, in (ii) or (ii.2), the compression, preferably the mechanical compression, more preferably the mechanical compression in a mechanical compression unit MC is done in at least two compression stages, preferably in at least two to ten compression stages, preferably in two to five compression stages, wherein the pressure of the heat transfer medium stream HTMS2 or HTMS2a respectively is increased from p₂ to at least one intermediate pressure level (p₂ₓ₋₁, p₂ₓ₋₂, . . . ) and subsequently to pressure p₃, wherein each intermediate pressure level is higher than p₂ and lower than p₃, and in cases of two or more intermediate pressure levels, higher than the preceding intermediate pressure level. For example, if there is one intermediate pressure level p₂ₓ between p₂ and p₃, then p₂ₓ is a pressure higher than p₂ and lower than p₃. In cases where there are at least two intermediate pressure levels p₂ₓ₋₁ and p₂ₓ₋₂, then p₂ₓ₋₁ is higher than p₂, but lower than p₂ₓ₋₂ and p₃, and p₂ₓ₋₂ is higher than p₂ and higher than p₂ₓ₋₁ but lower than p₃. In other words, the pressure is stepwise ramped-up from p₂ to p₃ via the intermediate pressure levels. Each pressure difference between p₂ and p₂ₓ₋₁, between p₂ₓ₋₁ and each optional further intermediate pressure level (p₂ₓ₋₂, . . . ) and between the intermediate pressure level preceding p₃ is at least 0.05 bar. Furthermore, in some embodiments with one or more intermediate pressure levels between p₂ and p₃, liquid heat transfer medium can be fed to the heat transfer medium stream HTMS2 or HTMS2a before the first (intermediate) compression step and/or before each subsequent (intermediate) compression step and/or before the final compression step, in order to avoid a heating-up of the heat transfer medium step due to the compression to a temperature above design temperature of the compressor. In embodiments with one or more intermediate pressure levels between p₂ and p₃, a partial heat transfer medium stream HTMS2x or HTMS2ax can be taken-off after the respective (intermediate) compression step. In some alternative embodiments, the take-off can also be conducted completely, i.e. that the complete heat transfer medium stream HTMS2x or HTMS2ax, which has a pressure level between p₂ and p₃ (p₂ₓ₋₁, p₂ₓ₋₂, . . . ), can be taken off and be optionally be put to a further use—in these alternative embodiments, no final compression up to p₃ is done, i.e. there is no heat transfer medium stream HTMS3 or HTMS3a respectively at the end of step (ii) or (ii.2).

### Heat Transfer Medium

In some preferred embodiments of the hydrogenation process, the heat transfer medium comprises water, preferably at least 90 weight-%, more preferably at least 95 weight-%, more preferably at least 99 weight-% of the heat transfer medium are water, each based on the total weight of the heat transfer medium being 100 weight-% In some preferred embodiments of the hydrogenation process, at least 90 weight-% of heat transfer medium stream HTMS1 are in liquid state and at least at least 90 weight-% of heat transfer medium stream HTMS2 and HTMS2a respectively are in gaseous state (H₂O_{gaseous}, water steam).

In some preferred embodiments of the hydrogenation process, HTMS2, HTMS2a, HTMS2x HTMS2xa, HTMS3 and/or HTMS3a, preferably HTMS3 and/or HTMS3a, each partially or totally, is/are used for reaching and/or maintaining the temperature necessary for carrying out the reaction in the reaction vessel, and/or for heating in a further process, which is preferably a process subsequent to the preparation of the compound having at least one amino group, more preferably a subsequent process, wherein the compound having at least one amino group is converted into a compound having at least one isocyanate group or a prior process, in which the compound comprising at least one nitro group is generated. Further the generated steam can be used in a steam net at other production plants for heating or can be sold to a third party.

### Reaction Vessel

In some preferred embodiments of the hydrogenation process, the reaction vessel comprised in the reaction apparatus comprises a reactor selected from the group consisting of (multi)tubular reactor, stirred tank reactor and loop reactor, wherein the reaction vessel is preferably a loop reactor. Regarding combinations of heat exchangers and reaction vessels, a person skilled in the art is aware that specific heat exchangers are mainly suitable for specific reactor types. For example, for a loop reactor, a heat exchanger located in the reaction vessel such as a shell and tube heat exchanger is used in some preferred embodiments.

### Reaction Conditions

The reactor is operated at a pressure in the range of from 5 to 100 bar, preferably in the range of from 10 to 50 bar, more preferably in the range of from 15 to 40 bar, more preferably in the range of from 20 to 30 bar, and the reaction mixture within the reactor has a temperature in the range of from 50 to 200° C., preferably in the range of from 60 to 180° C., more preferably in the range of from 70 to 150° C.

Preferably, the ratio of the compound having at least one nitro group to water in the reaction mixture is in the range from 10:1 to 1:10 (v/v), more preferably in the range from 4:1 to 1:1 (v/v), and the ratio of the compound having at least one nitro group/water mixture to at least one organic solvent is preferably in the range from 1000:1 to 1:1 (v/v), more preferably from 50:1 to 5:1 (v/v).]

### Heat Exchanger in Reactor

In some preferred embodiments of the hydrogenation process, heat exchanger HE is located within the reaction vessel.

### Heat Exchanger in Loop

In some alternative preferred embodiments of the hydrogenation process, the reaction apparatus comprises a reaction vessel and a circulation loop, preferably a circulation loop in fluid communication with the reaction vessel (loop reactor), and the heat exchanger HE is located within the circulation loop. “Fluid communication” regarding the circulation loop means that, as in a loop reactor, the reaction mixture (the reaction suspension) comprising the compound having at least one nitro group, optionally the corresponding compound having at least one amino group, a liquid medium, optionally hydrogen, and heterogeneous hydrogenation catalyst is pumped around or can be pumped around.

$$Reactor + cooling system$$

In some preferred embodiments of the hydrogenation process, the reaction apparatus comprises a reaction vessel and a cooling system in connection to the reaction vessel and a heat exchanger HE1 located within the reaction vessel or surrounding the reaction vessel at least partially, the reaction vessel preferably also comprising a circulation loop. Preferably, (i) then comprises
(i.1) transferring at least a part of the thermal energy generated in the reaction vessel to a cooling medium in HE1, preferably a cooling medium stream CMS1, obtaining a cooling medium stream CMS2, which has an increased thermal energy content compared to CMS1;
(i.2) transferring at least a part of the thermal energy comprised in cooling medium stream CMS2 to a heat transfer medium stream HTMS1 in a heat exchanger HE, thereby obtaining a heat transfer medium stream HTMS2, which has an increased thermal energy content compared to HTMS1.

Also heat exchanger HE1 is a heat exchanger, wherein the material streams are not in direct contact with each other but are rather spatially separated, so that there is only an exchange of thermal energy possible. Heat exchangers are generally known, wherein suitable heat exchangers for being arranged within the reaction vessel can be, for example, tubes through which a cooling medium stream flows, the direction of which is preferably parallel to the reactor wall, plate heat exchangers which preferably run parallel to the reactor wall, or also boiling tubes closed at the bottom, so-called field tubes, can be used. For any heat exchanger which comprises at least two compartments, which are spatially separated but are in thermal contact, it is interchangeably which material stream flows through or is in which compartment.

### Cooling Medium

In some preferred embodiments of the hydrogenation process, the cooling medium is selected from the group consisting of water, air, solvent and mixtures of two or more thereof, wherein the cooling medium preferably comprises water, preferably at least 90 weight-%, more preferably at least 95 weight-%, more preferably at least 99 weight-% of the heat transfer medium are water, each based on the total weight of the cooling medium being 100 weight-%. A solvent in the context of the cooling medium means any solvent having the same boiling conditions, especially the same boiling point as the solvent(s) used in the reaction mixture, wherein the “same” includes a deviation by ±5%. For example, if a C1 to C6 monoalcohol is used as solvent in the reaction mixture, especially ethanol and/or propanol, preferably iso-propanol, then a solvent having the same boiling conditions means a solvent having at 1013 mbar a boiling point in the range of from 78 to 86° C.

The reaction vessel is in some preferred embodiments a loop reactor. The loop reactor is a preferably vertical, preferably cylindrical, reactor. The loop reactor has a downward-facing jet nozzle arranged in the upper region of the reactor through which the starting materials and the reaction mixture are fed in, and has an outlet at any desired point of the reactor, preferably in the lower region, through which the reaction mixture is fed back to the jet nozzle in an external circuit by means of a conveying means, preferably a pump, and has flow reversal in the lower region of the reactor. The flow reversal and thus the formation of internal loop flow can be effected, in the case of take-off of the reaction mixture in the upper region of the reactor, by impact of the injected reaction mixture on the reactor base. In the case of the preferred take-off of the reaction mixture in the lower region of the reactor, the flow reversal is achieved by internals, in particular a baffle plate perpendicular to the reactor wall. The reactor contains one or more mixing chamber(s), which is/are preferably formed by cylindrical insertion tube(s) and is/are arranged within the reactor parallel to the reactor wall. The jet nozzle can be designed as a one- or two-component nozzle. In the case of the one-component nozzle, only the liquid reaction mixture is injected through the nozzle, and the (gaseous) hydrogen is fed into the reactor at any other desired point, but preferably at a point within the reaction mixture Suitable designs of a loop reactor are known and are, for example, described in WO 00/30743 A1, WO 00/35852 A1 or WO 2014/108352 A1. In some preferred embodiments, the loop reactor comprises a heat exchanger, preferably a heat exchanger located within the reactor. More preferably, said heat exchanger, equipped with suitable lines for introducing and removing a cooling medium stream, comprises one or more, preferably double-lined, tube(s) (field tubes), which are preferably arranged in the reactor substantially parallel to the reactor wall. “Substantially parallel” means that the tube(s) are arranged under an angle of ±30°, preferably ±20°, more preferably ±10°, with respect to the reactor wall. The cooling medium streams flows within the field tubes, which are surrounded on their outside by the reaction mixture in the reactor; a suitable setup is shown in FIG. 1 of WO 00/35852 A1. In some alternative embodiments, the reaction is conducted in tubes, whereas the cooling medium flow surrounds the tubes; a suitable setup is described shown in WO 2014/108352 A1.

The product, i.e. the compound having at least one amino group, is discharged from the system continuously or discontinuously, preferably continuously, at any desired point, but preferably at a point in the lower region of the reactor at its base or in particular from the external loop flow via a catalyst separation unit or without one. This separation unit can be a gravity separator, for example a settler, a suitable filter, for example a cross-flow filter, or a centrifuge. The catalyst can be separated from the product and then the catalyst can be fed back into the reactor system or discharged from the reactor system. The product is preferably discharged with retention of the catalyst. The product can then be purified by conventional and known methods, for example by distillation or extraction.

$$Loop reactor + Increase efficiency by increased temperatures ( serial setup )$$

In some preferred embodiments of the hydrogenation process, for the reaction apparatus comprising a reaction vessel and a cooling system in connection to the reaction vessel and a heat exchanger HE1 located within the reaction vessel or surrounding the reaction vessel at least partially, the process comprises:
(i.1.a) transferring at least a part of the thermal energy generated in the reaction vessel to a cooling medium, preferably a cooling medium stream CMS1, obtaining a cooling medium stream CMS2, which has an increased thermal energy content compared to CMS1;
(i.1.b) feeding CSM2 to a heat exchanger HE2 installed in the circulation loop of the loop reactor, thereby transferring a further part of the thermal energy generated in the reaction vessel to CMS2 and obtaining a cooling medium stream CMS4, which has an increased thermal energy content compared to CMS2;
(i.2) transferring at least a part of the thermal energy comprised in cooling medium stream CMS4 to a heat transfer medium stream HTMS1 in the heat exchanger HE, thereby obtaining a heat transfer medium stream HTMS2, which has an increased thermal energy content compared to HTMS1;
(ii) increasing the pressure of the heat transfer medium stream HTMS2 obtained in (i.2.a) having a pressure p₁, thereby obtaining a heat transfer medium stream HTMS3, which has an increased pressure p₂ compared to HTMS2.

In these embodiments, preferably a cooling medium stream CMS3 is obtained in (i.2.a), which has the same or a higher thermal energy content as CMS1, wherein preferably, CMS3 is reintroduced as CMS1 or at least as part thereof into the cooling system and the heat exchanger respectively.

$$Loop reactor + Increase efficiency by increased temperatures ( parallel setup )$$

In some alternative preferred embodiments of the hydrogenation process, for the reaction apparatus comprising a reaction vessel and a cooling system in connection to the reaction vessel and a heat exchanger HE1 located within the reaction vessel or surrounding the reaction vessel at least partially, the process comprises:
(i.1.a) transferring at least a part of the thermal energy generated in the reaction vessel to a cooling medium, preferably a cooling medium stream CMS1, obtaining a cooling medium stream CMS2, which has an increased thermal energy content compared to CMS1;
(i.1.b) obtaining a cooling medium stream CMS3 from heat exchanger HE, which has a decreased thermal energy content compared to CMS2;
(i.1.c) feeding at least a part of CMS3 (CMS3a) to a heat exchanger HE2 located in the circulation loop of the loop reactor, thereby transferring a further part of the thermal energy generated in the reaction vessel to CMS3a and obtaining a cooling medium stream CMS4, which has an increased thermal energy content compared to CMS3a;
(i.1.d) combining partial cooling medium stream CMS4 with cooling medium stream CMS2 thereby obtaining a combined cooling medium stream CMS2+CMS4;
(i.1.e) feeding the combined cooling medium stream CMS2+CMS4 to the heat exchanger HE;
(i.1.f) transferring at least a part of the thermal energy comprised in combined cooling medium stream CMS2+CMS4 to a heat transfer medium stream HTMS1 in the heat exchanger HE, thereby obtaining a heat transfer medium stream HTMS2, which has an increased thermal energy content compared to HTMS1;
(ii) increasing the pressure of the heat transfer medium stream HTMS2 obtained in (i.2.a) having a pressure p₁, thereby obtaining a heat transfer medium stream HTMS3, which has an increased pressure p₂ compared to HTMS2.

In these embodiments, preferably the cooling medium stream CMS3 obtained in (ii.a) has the same or a higher thermal energy content as CMS1, wherein preferably, at least a part of CMS3 (CSM3b) is reintroduced as CMS1 or at least as part thereof into the cooling system.

### Temperatures

In some preferred embodiments of the hydrogenation process, the cooling medium stream CMS2 or CMS4 or the combined stream of CSM2 and CSM4, which enters the heat exchanger HE has a temperature in the range of from 55 to 155° C. and the cooling medium stream CMS3, which leaves the heat exchanger HE has a temperature in the range of from 30 to 130° C.

### Energetic Balance

In some preferred embodiments of the hydrogenation process, at least 25% of the thermal energy generated in the reaction vessel are transferred, optionally via the cooling medium, to the heat transfer medium, preferably to heat transfer medium stream HTMS1 and heat transfer medium stream HTMS2 respectively.

In some preferred embodiments of the hydrogenation process, in case that the heat transfer medium stream HTMS2 is water steam (H₂O_{gaseous}), the ratio of the amount of steam generated in tons to the amount of thermal energy generated (therm.en.gen) in the reaction vessel in MW is in the range of from 1.5 to 2.5 tₛₜₑₐₘ/MW_{therm.en.gen}.

In some alternative preferred embodiments of the hydrogenation process, in case that the heat transfer medium stream HTMS2 is water steam (H₂O_{gaseous}) the ratio of electrical power demand in MWₑₗ to the amount of steam generated in tons is in the range from 0.025 to 0.3 MWₑₗ/tₛₜₑₐₘ, preferably in the range of from 0.075 to 0.25 MWₑₗ/tₛₜₑₐₘ

### Compound to be Hydrogenated

The compound to be hydrogenated is preferably a compound having at least one nitro group. In some embodiments of the hydrogenation process, the compound having at least one nitro group (aka the “nitro compound”) is preferably an organic compound having at least one nitro group, preferably selected from the group of nitro alcohol, nitroaromatic and mixtures of nitroalcohol and nitroaromatic.

The nitro compound is preferably an organic compound having at least one nitro group, preferably selected from the group of nitro alcohol, nitroaromatic and mixtures of nitroalcohol and nitroaromatic. In preferred embodiments, the nitro compound for hydrogenation is a nitroaromatic, preferably selected from the group consisting of mononitroaromatic, dinitroaromatic, polynitroaromatic and mixtures of two or more thereof. A “mononitroarimatic” is an aromatic compound having only one nitro group as substituent. A “dinitroaromatic” is an aromatic compound having two nitro groups as substituents. A “polynitroaromatic” in the context of the invention is an aromatic compound having at least three nitro groups. In some preferred embodiments, the compound having at least one nitro group is selected from the group consisting of mononitroaromatic, dinitroaromatic and mixtures of mononitroaromatic and dinitroaromatic, preferably the compound having at least one nitro group comprises at least a dinitroaromatic. A mononitroaromatic is preferably an aromatic compound having in the range of from 6 to 18 carbon atoms and one nitro group as substituent. In some preferred embodiments, the mononitroaromatic is selected from the group consisting of mononitrotoluene, halogen derivatives of mononitrotoluene, mononitrobenzene, the halogen derivatives of mononitrobenzene, mononitroxylene, mononitronaphthalene, nitroaniline and mixtures of two or more thereof. Preferably, the mononitroaromatic is selected from the group consisting of nitrobenzene, o-nitrotoluene, m-nitrotoluene, p-nitrotoluene, 1,2-dimethyl-3-nitrobenzene, 1,2-dimethyl-4-nitrobenzene, 1,4-dimethyl-2-nitrobenzene, 1,3-dimethyl-2-nitrobenzene, 2,4-dimethyl-1-nitrobenzene, 1,3-dimethyl-5-nitrobenzene, 1-nitronaphthalene, 2-nitronaphthalene, o-chloronitrobenzene, m-chloronitrobenzene, p-chloronitrobenzene, 1,2-dichloro-4-nitrobenzene, 1,4-dichloro-2-nitrobenzene, 2,4-dichloro-1-nitrobenzene, 1,2-dichloro-3-nitrobenzene, 4-chloro-2-nitrotoluene, 4-chloro-3-nitrotoluene, 2-chloro-4-nitrotoluene, 2-chloro-6-nitrotoluene, o-nitroaniline, m-nitroaniline, p-nitroaniline and mixtures of two or more thereof. In some preferred embodiments, the mononitroaromatic is selected from the group consisting of mononitrobenzene, halogenated mononitrobenzene, mononitrotoluene, and mixtures of two or more thereof, more preferably selected from the group consisting of nitrobenzene, o-nitrotoluene, m-nitrotoluene, p-nitrotoluene, 1,2-dimethyl-3-nitrobenzene, 1,2-dimethyl-4-nitrobenzene, 1,4-dimethyl-2-nitrobenzene, 1,3-dimethyl-2-nitrobenzene, 2,4-dimethyl-1-nitrobenzene, 1,2-dichloro-4-nitrobenzene, 1,4-dichloro-2-nitrobenzene, 2,4-dichloro-1-nitrobenzene, 1,2-dichloro-3-nitrobenzene and mixtures of two or more thereof, more preferably from the group consisting of o-nitrotoluene, m-nitrotoluene, p-nitrotoluene and mixtures of two or more thereof. A dinitroaromatic is preferably an aromatic compound having in the range of from 6 to 18 carbon atoms. Preferably, the dinitroaromatic is selected from the group consisting of dinitrotoluene, halide of dinitrotoluene dinitrobenzene, halide of dinitrobenzene, dinitronaphthalene and mixtures of two or more thereof. In some preferred embodiments, the dinitroaromatic is selected from the group consisting of 1,2-dinitrobenzene, 1,3-dinitrobenzene, 1,4-dinitrobenzene, 2,3-dinitrotoluene, 2,4-dinitrotoluene, 3,4-dinitrotoluene, 3,5-dintritotoluene, 2,6-dinitrotoluene, 3,6-dinitrotoluene, and mixtures of two or more thereof, more preferably from the group consisting of 2,3-dinitrotoluene, 2,4-dinitrotoluene, 3,4-dinitrotoluene, 3,5-dintritotoluene, 2,6-dinitrotoluene, 3,6-dinitrotoluene, and mixtures of two or more thereof.]

In some preferred embodiments of the hydrogenation process, the compound having at least one nitro group comprises 2,4-dinitrotoluene, 2,6-dinitrotoluene or a mixture of 2,4-dinitrotoluene and 2,6-dinitrotoluene. In some preferred embodiments, the compound having at least one nitro group comprises 2,4-dinitrotoluene, 2,6-dinitrotoluene or a mixture of 2,4-dinitrotoluene and 2,6-dinitrotoluene. Industrial mixtures comprising 2,4-dinitrotoluene and 2,6-dinitrotoluene are also suitable, wherein these mixtures preferably comprise at least 55 weight-% 2,4-dinitrotoluene and up to 35 weight-% of 2,6-dinitrotoluene with proportions of preferably up to 5 weight-% of vicinal dinitrotoluene and preferably up to 1.5 weight-% of 2,5- and 3,5-dinitrotoluene based on the overall mixture being 100 weight-%.

The above-mentioned nitro alcohol(s) and nitroaromatic(s) are commercially available. The nitro alcohols and nitroaromatics used may furthermore be obtained by chemical synthesis, such as dinitrotoluenes may be obtained by nitration of toluene for example. The thus formed reaction product usually comprises not only the desired nitro compound but also numerous impurities.

Provided that an above described mixture is employed the weight ratio of an amine compound to water is preferably in the range from 10:1 to 1:10, preferably in the range from 8:1 to 1:5 and particularly preferably in the range from 4:1 to 1:3 and the weight ratio of the amine/water mixture to C1 to C6 monoalcohol is preferably 1000:1 to 1:1, preferably 500:1 to 2.5:1 and particularly preferably 50:1 to 5:1.

The amount of the employed alcoholic solvent, i.e. the C1 to C6 monoalcohol, and of the catalyst-reactivating additions is not restricted in any particular way in the context of the process according to the invention and may be chosen freely as required.

The process according to the invention for hydrogenation of nitro compounds to the corresponding amines may additionally be performed in the absence of solvents. In this procedure the workup of the reaction mixture after the hydrogenation is simplified and side reactions with the solvent are moreover completely inhibited.

### Hydrogen (H₂)

Any gas free of harmful amounts of catalyst poisons such as CO, which contains free hydrogen can be used as hydrogenation gas, for example, a reformer exhaust gas can be used or a mixture of hydrogen with nitrogen and/or carbon monoxid. Preferably, pure hydrogen, i.e. hydrogen having a purity of at least 90%, is used as hydrogenation gas or a mixture of hydrogen and an inert gas, preferably nitrogen, can be used as hydrogenation gas, wherein at least 90 weight-% of the mixture consist of hydrogen and nitrogen, the total weight of the mixture being 100 weight-%.

### Solvent(s)

The conversion of the compound having at least one nitro group into the corresponding compound having at least one amino group is done (within the reactor) in a liquid medium, preferably in a solution or suspension comprising water and optionally one or more C1 to C6 mono alcohol, preferably a C1 to C6 mono alcohol from the group consisting of C1 to C5 mono alcohols, more preferably selected from the group consisting of methanol, ethanol, propanol, including n-propanol and iso-propanol, and mixtures of two or three thereof, more preferably the solvent contains at least iso-propanol. A catalyst-reactivating additive, preferably selected from the group consisting of aprotic solvent, more preferably selected from the group consisting of DMF, dioxane, THF or a mixture of two or more thereof, is used in some embodiments.

### Reaction Conditions

The reactor is operated at a pressure in the range of from 5 to 100 bar, preferably in the range of from 10 to 50 bar, more preferably in the range of from 15 to 40 bar, more preferably in the range of from 20 to 30 bar, and the reaction mixture within the reactor has a temperature in the range of from 50 to 200° C., preferably in the range of from 60 to 180° C., more preferably in the range of from 70 to 150° C.

### Catalyst

The catalyst used in the reaction is preferably a hydrogenation catalyst. Suitable hydrogenation catalysts are known per se for aromatic nitro compounds. It is possible to use homogeneous and/or heterogeneous catalysts, wherein the use of heterogenous catalysts is preferred. The heterogeneous catalysts are employed in the form of particles and are suspended in the reaction suspension. Suitable catalysts are metals from sub-group VIII of the Periodic Table, which are preferably supported on support materials such as activated carbon or oxides of aluminum, silicon or other materials. Preference is given to Raney nickel and/or supported catalysts based on nickel, copper, palladium and/or platinum. Suitable hydrogenation catalysts are known, for example, from WO 00/35852 A1 or WO 2005/037768 A1. The hydrogenation catalyst is preferably used in an amount of 0.01 to 10, preferably 0.1 to 5, particularly preferably 0.2 to 2% by weight, based on the weight of the reaction mixture being 100% by weight.

### 2^{nd} Aspect—Reaction System/Reaction Apparatus

A second aspect of the invention is directed to a reaction system for hydrogenation, preferably for converting a compound having at least one nitro group into the corresponding compound having at least one amino group by hydrogenation, comprising:
(a) a reactor having
   (a.1) means for feeding gaseous and liquid materials into the reactor;
   (a.2) means for intermixing in the reactor,
   (a.3) an outlet for removing reaction mixture from the reactor;
   (a.4) at least one inlet for reintroduction of the reaction mixture;
   (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
   (a.6) at least one heat exchanger HE1 arranged within the reactor, outside of the reactor or in the circuit lines (a.5), wherein the heat exchanger HE1 has at least one inlet (a.6.1) for feeding a cooling medium into the heat exchanger and at least one outlet (a.6.2) for removing the cooling medium from the heat exchanger HE1;
(b) a heat exchanger HE comprising
   (b.1) at least one inlet (b.1.1) for feeding a cooling medium into the heat exchanger HE, and at least one outlet (b.1.2) for removing the cooling medium from the exchanger HE;
   (b.2) at least one inlet (b.2.1) for feeding a heat transfer medium stream into the heat exchanger HE, and at least one outlet (b.2.2) for removing the heat transfer medium stream from the heat exchanger HE;
   wherein the heat exchanger HE is built to allow transfer of thermal energy from a cooling medium stream to a heat transfer medium stream;
(c) lines in fluid connection with the at least one heat exchanger HE1 (a.6) and the heat exchanger HE (b), which are built and arranged
   (c.1) to allow transfer of the cooling medium stream coming from the at least one outlet of the heat exchanger HE1 (a.6.2) into an inlet of the heat exchanger HE (b.1.1) and reintroduction of the cooling medium stream coming from the at least one outlet (b.1.2) for removing the cooling medium from the heat exchanger HE to the at least one inlet (a.6.1) for feeding a cooling medium into the heat exchanger HE1; and
   (c.2) to allow introduction of a heat transfer medium stream into the at least one inlet (b.2.1) of the heat exchanger HE for feeding a heat transfer medium stream into the heat exchanger HE and removal of a heat transfer medium stream from the at least one outlet (b.2.2) of the heat exchanger HE for removing the heat transfer medium stream of the heat exchanger HE;
(d) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.

In some alternative embodiments, the reaction system for hydrogenation, preferably for converting a compound having at least one nitro group into the corresponding compound having at least one amino group by hydrogenation, comprises:
(a) a reactor having
   (a.1) means for feeding gaseous and liquid materials into the reactor;
   (a.2) means for intermixing in the reactor,
   (a.3) an outlet for removing reaction mixture from the reactor;
   (a.4) at least one inlet for reintroduction of the reaction mixture;
   (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
(b) a heat exchanger HE comprising
   (b.1) at least one inlet (b.1.1) for feeding the reaction mixture into the heat exchanger HE, and at least one outlet (b.1.2) for removing the reaction mixture from the exchanger HE;
   (b.2) at least one inlet (b.2.1) for feeding a heat transfer medium stream into the heat exchanger HE, and at least one outlet (b.2.2) for removing the heat transfer medium stream from the heat exchanger HE;
   wherein the heat exchanger HE is built to allow transfer of thermal energy from a reaction mixture to a heat transfer medium stream;
(d) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.

Preferably, in the preferred embodiments or preferred alternative embodiments above, the reaction system further comprises:
(x) means for increasing the temperature of the heat transfer medium stream coming from the heat exchanger HE (HTMS2), which has a temperature T₂, to a temperature T₂ₐ, which is higher than T₂, thereby obtaining a heat transfer medium stream (HTMS2a) having a pressure p₂ₐ, which is in the range of from p₂ to p₂−300 mbar, more preferably p₂ₐ is in the range of from p₂ to p₂−100 mbar and a temperature T₂ₐ; wherein the means (x) for increasing the temperature are preferably one or more overheaters OH.

In some preferred embodiments of the reaction system, the means (x) are preferably installed between (b) and (d), wherein preferably the means (d) comprise:
(x.1) an inlet (x.1) for feeding the heat transfer medium stream HTM2 to the means (x), (x.2) an outlet (x.2) for removing heat transfer medium stream HTM2a from the means (x), wherein preferably the inlet (x.1) is connected to the at least one outlet (b.2.2) of heat exchanger HE, and an outlet (x.2) of the means (x) is connected to an inlet (d.1) of the means (d) for compression, wherein the connections are built and arranged to allow transfer of the heat transfer medium stream HTMS2 from the at least one outlet (b.2.2) of heat exchanger HE to the inlet (x.1) of the means (x) and to allow transfer of the heat transfer medium stream HTMS2a from the outlet (x.2) to an inlet of the means for compression (d).

In some preferred embodiments of the reaction system, the means for compression according to (d) are one or more mechanical compression units, preferably one or more mechanical compressor(s).

In some preferred embodiments of the reaction system, two or more mechanical compression units, preferably two or more mechanical compressor(s) (MC1, MC2, . . . ) are arranged in series and are connected by lines allowing transfer of a compressed heat transfer medium stream.

In some preferred embodiments of the reaction system, at least one outlet is installed between two consecutive mechanical compression units, wherein the outlet is built and arranged for allowing a total or partial take-off of heat transfer medium stream HTMS2x or HTMS2ax, which has a pressure level between p₂ and p₃ (p₂ₓ₋₁, p₂ₓ₋₂, . . . ).

All details, embodiments and preferred embodiments disclosed above in the section related to the first aspect apply also for the reaction system, i.e. the second aspect of the invention.

The present invention is further illustrated by the following embodiments and combinations of embodiments as indicated by the respective dependencies and back-references. In particular, it is noted that in each instance where a range of embodiments is mentioned, for example in the context of a term such as “The . . . of any of embodiments 1 to 4”, every embodiment in this range is meant to be explicitly disclosed for the skilled person, i.e. the wording of this term is to be understood by the skilled person as being synonymous to “The . . . of any of embodiments 1, 2, 3, and 4”.
1. A hydrogenation process for the conversion of a compound comprising reacting the compound in a liquid medium with hydrogen in the presence of a heterogeneous hydrogenation catalyst in a reaction vessel comprised in a reaction apparatus, the process comprising
   (i) transferring at least a part of the thermal energy generated in the reaction vessel to a heat transfer medium in a heat exchanger HE, preferably to a heat transfer medium stream HTMS1, obtaining a heat transfer medium stream HTMS2, which has an increased thermal energy content compared to HTMS1;
   (ii) increasing the pressure of the heat transfer medium stream HTMS2 having a pressure p₂, thereby obtaining a heat transfer medium stream HTMS3, which has an increased pressure p₃ compared to HTMS2.
2. The hydrogenation process of embodiment 1, being a process for the hydrogenation of a compound having at least one nitro group into the corresponding compound having at least one amino group comprising reacting the compound having at least one nitro group in a liquid medium with hydrogen in the presence of a heterogeneous hydrogenation catalyst in a reaction vessel.
3. The hydrogenation process of embodiment 1 or 2, wherein the pressure difference between p₃ and p₂ [Δp(p₃−p₂)] is in the range of from 1 to 40 bar.
4. The hydrogenation process of any one of embodiments 1 to 3, wherein heat transfer medium stream HTMS2 has a pressure p₂ in the range of from −0.9 to 3 barg, preferably in the range of from −0.7 barg to 1 barg.
5. The hydrogenation process of any one of embodiments 1 to 4, wherein heat transfer medium stream HTMS3 has a pressure p₃ in the range of from 0.1 to 40 barg, preferably in the range of from 3 barg to 20 barg.
6. The hydrogenation process of any one of embodiments 1 to 5, wherein increasing the pressure of the heat transfer medium stream HTMS2 having a pressure p₂ in (ii) is done by mechanical compression, preferably in a mechanical compression unit MC.
7. The hydrogenation process of any one of embodiments 1 to 6, wherein (ii) comprises
   (ii.1) increasing the temperature of heat transfer medium stream HTMS2 having a temperature T₂ to a temperature T₂ₐ, which is higher than T₂, preferably in an overheater OH, thereby obtaining a heat transfer medium stream HTMS2a having a pressure p₂ₐ and a temperature T₂ₐ;
   (ii.2) compressing HTMS2a, thereby obtaining a heat transfer medium stream HTMS3a, which has an increased pressure p₃ₐ compared to HTMS2 and HTMS2a respectively and a temperature T₃ₐ.
8. The hydrogenation process of embodiment 7, wherein T₂ₐ is in the range of from the temperature at the boiling point (T_{BP}) of the heat transfer medium at a pressure p₂ₐ+3 K to temperature at the boiling point of the heat transfer medium (T_{BP}) at a pressure p₂ₐ+40 K and/or T₃ₐ is ≥the temperature at the boiling point (T_{BP}) of the heat transfer medium at a pressure p₃ₐ+3 K.
9. The hydrogenation process of any one of embodiments 1 to 8, wherein in (ii) or (ii.2), the compression, preferably the mechanical compression, more preferably the mechanical compression in a mechanical compression unit MC is done in one compression stage, wherein the pressure of the heat transfer medium stream HTMS2 or HTMS2a respectively is increased from p₁ to p₂.
10. The hydrogenation process of any one of embodiments 1 to 8, wherein in (ii) or (ii.2), the compression, preferably the mechanical compression, more preferably the mechanical compression in a mechanical compression unit MC is done in at least two compression stages, preferably in at least two to ten compression stages, preferably in two to five compression stages, wherein the pressure of the heat transfer medium stream HTMS2 or HTMS2a respectively is increased from p₂ to at least one intermediate pressure level (p₂ₓ₋₁, p₂ₓ₋₂, . . . ) and subsequently to pressure p₃, wherein each intermediate pressure level is higher than p₂ and lower than p₃, and in cases of two or more intermediate pressure levels, higher than the preceding intermediate pressure level.
11. The hydrogenation process of any one of embodiments 1 to 10, wherein the heat transfer medium comprises water, preferably at least 90 weight-%, more preferably at least 95 weight-%, more preferably at least 99 weight-% of the heat transfer medium are water, each based on the total weight of the heat transfer medium being 100 weight-%
12. The hydrogenation process of embodiment 11, wherein at least 90 weight-% of heat transfer medium stream HTMS1 are in liquid state and at least at least 90 weight-% of heat transfer medium stream HTMS2 and HTMS2a respectively are in gaseous state (H₂O_{gaseous}, water steam).
13. The hydrogenation process of any one of embodiments 1 to 12, wherein HTMS2, HTMS2a, HTMS2x HTMS2xa, HTMS3 and/or HTMS3a, preferably HTMS3 and/or HTMS3a, each partially or totally, is/are used for reaching and/or maintaining the temperature necessary for carrying out the reaction in the reaction vessel, and/or for heating in a further process, which is preferably a process subsequent to the preparation of the compound having at least one amino group, more preferably a subsequent process, wherein the compound having at least one amino group is converted into a compound having at least one isocyanate group.
14. The hydrogenation process of any one of embodiments 1 to 13, wherein the reaction vessel comprised in the reaction apparatus comprises a reactor selected from the group consisting of (multi)tubular reactor, stirred tank reactor and loop reactor, wherein the reaction vessel is preferably a loop reactor.
15. The hydrogenation process of embodiment 14, wherein heat exchanger HE is located within the reaction vessel.
16. The hydrogenation process of embodiment 14, wherein the reaction apparatus comprises a reaction vessel and a circulation loop, preferably a circulation loop in fluid communication with the reaction vessel (loop reactor), and the heat exchanger HE is located within the circulation loop.
17. The hydrogenation process of embodiment 14, wherein the reaction apparatus comprises a reaction vessel and a cooling system in connection to the reaction vessel and a heat exchanger HE1 located within the reaction vessel or surrounding the reaction vessel at least partially, the reaction vessel preferably also comprising a circulation loop.
18. The hydrogenation process of embodiment 17, wherein (i) comprises
   (i.1) transferring at least a part of the thermal energy generated in the reaction vessel to a cooling medium in HE1, preferably a cooling medium stream CMS1, obtaining a cooling medium stream CMS2, which has an increased thermal energy content compared to CMS1;
   (i.2) transferring at least a part of the thermal energy comprised in cooling medium stream CMS2 to a heat transfer medium stream HTMS1 in a heat exchanger HE, thereby obtaining a heat transfer medium stream HTMS2, which has an increased thermal energy content compared to HTMS1.
19. The hydrogenation process of embodiment 17 or 18, wherein the cooling medium is selected from the group consisting of water, air, solvent and mixtures of two or more thereof, wherein the cooling medium preferably comprises water, preferably at least 90 weight-%, more preferably at least 95 weight-%, more preferably at least 99 weight-% of the heat transfer medium are water, each based on the total weight of the cooling medium being 100 weight-%.
20. The hydrogenation process of any one of embodiments 17 to 19, wherein for the reaction apparatus comprising a reaction vessel and a cooling system in connection to the reaction vessel and a heat exchanger HE1 located within the reaction vessel or surrounding the reaction vessel at least partially, the process comprises:
   (i.1.a) transferring at least a part of the thermal energy generated in the reaction vessel to a cooling medium, preferably a cooling medium stream CMS1, obtaining a cooling medium stream CMS2, which has an increased thermal energy content compared to CMS1;
   (i.1.b) feeding CSM2 to a heat exchanger HE2 installed in the circulation loop of the loop reactor, thereby transferring a further part of the thermal energy generated in the reaction vessel to CMS2 and obtaining a cooling medium stream CMS4, which has an increased thermal energy content compared to CMS2;
   (i.2) transferring at least a part of the thermal energy comprised in cooling medium stream CMS4 to a heat transfer medium stream HTMS1 in the heat exchanger HE, thereby obtaining a heat transfer medium stream HTMS2, which has an increased thermal energy content compared to HTMS1;
   (ii) increasing the pressure of the heat transfer medium stream HTMS2 obtained in (i.2.a) having a pressure p₁, thereby obtaining a heat transfer medium stream HTMS3, which has an increased pressure p₂ compared to HTMS2.
21. The hydrogenation process of embodiment 20, wherein the a cooling medium stream CMS3 is obtained in (i.2.a), which has the same or a higher thermal energy content as CMS1, wherein preferably, CMS3 is reintroduced as CMS1 or at least as part thereof into the cooling system and the heat exchanger respectively.
22. The hydrogenation process of any one of embodiments 17 to 19, wherein for the reaction apparatus comprising a reaction vessel and a cooling system in connection to the reaction vessel and a heat exchanger HE1 located within the reaction vessel or surrounding the reaction vessel at least partially, the process comprises:
   (i.1.a) transferring at least a part of the thermal energy generated in the reaction vessel to a cooling medium, preferably a cooling medium stream CMS1, obtaining a cooling medium stream CMS2, which has an increased thermal energy content compared to CMS1;
   (i.1.b) obtaining a cooling medium stream CMS3 from heat exchanger HE, which has a decreased thermal energy content compared to CMS2;
   (i.1.c) feeding at least a part of CMS3 (CMS3a) to a heat exchanger HE2 located in the circulation loop of the loop reactor, thereby transferring a further part of the thermal energy generated in the reaction vessel to CMS3a and obtaining a cooling medium stream CMS4, which has an increased thermal energy content compared to CMS3a;
   (i.1.d) combining partial cooling medium stream CMS4 with cooling medium stream CMS2 thereby obtaining a combined cooling medium stream CMS2+CMS4;
   (i.1.e) feeding the combined cooling medium stream CMS2+CMS4 to the heat exchanger HE;
   (i.1.f) transferring at least a part of the thermal energy comprised in combined cooling medium stream CMS2+CMS4 to a heat transfer medium stream HTMS1 in the heat exchanger HE, thereby obtaining a heat transfer medium stream HTMS2, which has an increased thermal energy content compared to HTMS1;
   (ii) increasing the pressure of the heat transfer medium stream HTMS2 obtained in (i.2.a) having a pressure p₁, thereby obtaining a heat transfer medium stream HTMS3, which has an increased pressure p₂ compared to HTMS2.
23. The hydrogenation process of embodiment 22, wherein the cooling medium stream CMS3 obtained in (ii.a) has the same or a higher thermal energy content as CMS1, wherein preferably, at least a part of CMS3 (CSM3b) is reintroduced as CMS1 or at least as part thereof into the cooling system.
24. The hydrogenation process of any one of embodiments 17 to 23, wherein the cooling medium stream CMS2 or CMS4 or the combined stream of CSM2 and CSM4, which enters the heat exchanger HE has a temperature in the range of from 55 to 155° C. and the cooling medium stream CMS3, which leaves the heat exchanger HE has a temperature in the range of from 30 to 130° C.
25. The hydrogenation process of any one of embodiments 1 to 24, wherein at least 25% of the thermal energy generated in the reaction vessel are transferred, optionally via the cooling medium, to the heat transfer medium, preferably to heat transfer medium stream HTMS1 and heat transfer medium stream HTMS2 respectively.
26. The hydrogenation process of any one of embodiments 1 to 25, wherein in case that the heat transfer medium stream HTMS2 is water steam (H₂O_{gaseous}), the ratio of the amount of steam generated in tons to the amount of thermal energy generated in the reaction vessel in MW is in the range of from 1.5 to 2.5 tₛₜₑₐₘ/MW_{therm.en.gen}.
27. The hydrogenation process of embodiment 26, wherein in case that the heat transfer medium stream HTMS2 is water steam (H₂O_{gaseous}) the ratio of electrical power demand in MWₑₗ to the amount of steam generated in tons is in the range from 0.025 to 0.3 MWₑₗ/tₛₜₑₐₘ, preferably in the range of from 0.075 to 0.25 MWₑₗ/tₛₜₑₐₘ
28. The hydrogenation process of any one of embodiments 1 to 27, wherein the compound having at least one nitro group (aka the “nitro compound”) is preferably an organic compound having at least one nitro group, preferably selected from the group of nitro alcohol, nitroaromatic and mixtures of nitroalcohol and nitroaromatic.
29. The hydrogenation process of embodiment 28, wherein the compound having at least one nitro group comprises 2,4-dinitrotoluene, 2,6-dinitrotoluene or a mixture of 2,4-dinitrotoluene and 2,6-dinitrotoluene.
30. A reaction system for hydrogenation, preferably for converting a compound having at least one nitro group into the corresponding compound having at least one amino group by hydrogenation, comprising:
   (a) a reactor having
      (a.1) means for feeding gaseous and liquid materials into the reactor;
      (a.2) means for intermixing in the reactor,
      (a.3) an outlet for removing reaction mixture from the reactor;
      (a.4) at least one inlet for reintroduction of the reaction mixture;
      (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
      (a.6) at least one heat exchanger HE1 arranged within the reactor, outside of the reactor or in the circuit lines (a.5), wherein the heat exchanger HE lhas at least one inlet (a.6.1) for feeding a cooling medium into the heat exchanger and at least one outlet (a.6.2) for removing the cooling medium from the heat exchanger HE1;
   (b) a heat exchanger HE comprising
      (b.1) at least one inlet (b.1.1) for feeding a cooling medium into the heat exchanger HE, and at least one outlet (b.1.2) for removing the cooling medium from the exchanger HE;
      (b.2) at least one inlet (b.2.1) for feeding a heat transfer medium stream into the heat exchanger HE, and at least one outlet (b.2.2) for removing the heat transfer medium stream from the heat exchanger HE;
      wherein the heat exchanger HE is built to allow transfer of thermal energy from a cooling medium stream to a heat transfer medium stream;
   (c) lines in fluid connection with the at least one heat exchanger HE1 (a.6) and the heat exchanger HE (b), which are built and arranged
      (c.1) to allow transfer of the cooling medium stream coming from the at least one outlet of the heat exchanger HE1 (a.6.2) into an inlet of the heat exchanger HE (b.1.1) and reintroduction of the cooling medium stream coming from the at least one outlet (b.1.2) for removing the cooling medium from the heat exchanger HE to the at least one inlet (a.6.1) for feeding a cooling medium into the heat exchanger HE1; and
      (c.2) to allow introduction of a heat transfer medium stream into the at least one inlet (b.2.1) of the heat exchanger HE for feeding a heat transfer medium stream into the heat exchanger HE and removal of a heat transfer medium stream from the at least one outlet (b.2.2) of the heat exchanger HE for removing the heat transfer medium stream of the heat exchanger HE;
   (d) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.
31. A reaction system for hydrogenation, preferably for converting a compound having at least one nitro group into the corresponding compound having at least one amino group by hydrogenation, comprising:
   (a) a reactor having
      (a.1) means for feeding gaseous and liquid materials into the reactor;
      (a.2) means for intermixing in the reactor,
      (a.3) an outlet for removing reaction mixture from the reactor;
      (a.4) at least one inlet for reintroduction of the reaction mixture;
      (a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
   (b) a heat exchanger HE comprising
      (b.1) at least one inlet (b.1.1) for feeding the reaction mixture into the heat exchanger HE, and at least one outlet (b.1.2) for removing the reaction mixture from the exchanger HE;
      (b.2) at least one inlet (b.2.1) for feeding a heat transfer medium stream into the heat exchanger HE, and at least one outlet (b.2.2) for removing the heat transfer medium stream from the heat exchanger HE;
      wherein the heat exchanger HE is built to allow transfer of thermal energy from a reaction mixture to a heat transfer medium stream;
   (d) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.
32. The reaction system according to embodiment 30 or 31, further comprising
   (x) means for increasing the temperature of the heat transfer medium stream coming from the heat exchanger HE (HTMS2), which has a temperature T₂, to a temperature T₂ₐ, which is higher than T₂, thereby obtaining a heat transfer medium stream (HTMS2a) having a pressure in the range of from p₂−300 mbar to p₂ preferably in the range of from p₂−100 mbar to p₂, and a temperature T₂ₐ;
      wherein the means (x) for increasing the temperature are preferably one or more overheaters OH.
33. The reaction system according to embodiment 32, wherein the means (x) are preferably installed between (b) and (d), wherein preferably the means (x) comprise:
   (x.1) an inlet (x.1) for feeding the heat transfer medium stream HTM2 to the means (x),
   (x.2) an outlet (x.2) for removing heat transfer medium stream HTM2a from the means (x),
   wherein preferably the inlet (x.1) is connected to the at least one outlet (b.2.2) of heat exchanger HE, and an outlet (x.2) of the means (x) is connected to an inlet (d.1) of the means (d) for compression, wherein the connections are built and arranged to allow transfer of the heat transfer medium stream HTMS2 from the at least one outlet (b.2.2) of heat exchanger HE to the inlet (x.1) of the means (x) and to allow transfer of the heat transfer medium stream HTMS2a from the outlet (x.2) to an inlet of the means for compression (d).
34. The reaction system according to any one of embodiments 30 to 33, wherein the means for compression according to (d) are one or more mechanical compression units, preferably one or more mechanical compressor(s).
35. The reaction system according to any one of embodiments 30 to 34, wherein two or more mechanical compression units, preferably two or more mechanical compressor(s) (MC1, MC2, . . . ) are arranged in series and are connected by lines allowing transfer of a compressed heat transfer medium stream.
36. The reaction system according to any one of embodiments 30 to 35, wherein at least one outlet is installed between two consecutive mechanical compression units, wherein the outlet is built and arranged for allowing a total or partial take-off of heat transfer medium stream HTMS₂ₓ or HTMS₂ₐₓ, which has a pressure level between p₂ and p₃ (p₂ₓ₋₁, p₂ₓ₋₂, . . . ).

The present invention is further illustrated by the following reference examples, comparative examples, and examples.

### EXAMPLES

### Simulations

All simulations were done with the process simulation software ASPEN PLUS™ v.8.6 or Ebsilon®, in combination with excel-based estimation calculation with specified quality grade. The components used in the process simulation and their characteristics respectively, were taken from the ASPEN PLUS™ v.8.6 PURE32 Database.

### Definitions, Abbreviations

Pressures indicated in “barg” are related to gauge pressure. Since gauge pressure is measured against the ambient pressure, each pressure value (or range) indicated in barg it is equal to absolute pressure minus atmospheric pressure.

### Comparative Example 1: Hydrogenation of Dinitro Toluene with Primary and Secondary Cooling Medium Loops

The hydrogenation of dinitro toluene (mixture of 2,4-dinitrotoluene and 2,6-dinitrotoluene, about 80:20 (w/w)) to toluene diamine was performed in a hydrogenation reactor, which was a loop reactor. To secure a good mixing the reactor content, i.e. the hydrogenation bath, which mainly consisted of water, solvent, solid catalyst and reactands dinitro toluene as well as products toluene diamine was pumped around by an external loop. The reaction heat in the reactor was removed from the hydrogenation bath by a primary cooling medium loop containing a primary cooling medium. The warm primary cooling medium was cooled down back by a secondary cooling loop, which was operated with a second cooling medium (for example, river water or air cooler) and the cold primary cooling medium was then sent back to the reactor.

The thermal energy released from the reaction, which had to be taken on by the cooling medium(s), was 40 MW.

### Example 1: Hydrogenation of Dinitro Toluene with Heat Exchanger in the Reactor

A hydrogenation of dinitro toluene was carried out as in Comparative Example 1. Contrary to Comparative Example 1, no primary or secondary cooling medium loop was operated. Instead, a set-up as shown in FIG. 1 was used, wherein a heat exchanger, here a steam generator, located in the reactor was used to recover (at least partially) the reaction heat, i.e. the thermal energy, which was initially transferred to heat transfer medium in the heat exchanger. The reaction heat of the hydrogenation reaction warmed up the heat exchanger medium (boiling water) so that in the evaporator part of the steam generator, steam was generated, having a pressure in the range of from −0.9 barg to 3 barg, preferably in the range of from −0.7 barg to 1 barg (low pressure steam). The low pressure steam was then, optionally after warm-up in an overheater, subsequently pressurized by mechanical vapor compression to a pressure in the range of from 2 barg to 40 barg, preferably in the range of from 3 barg to 20 barg. A backup cooling system was installed with a separate cooling loop inside of the reactor or a further heat exchanger in the hydrogenation bath circulation loop to remove the reaction heat in case of a shutdown of the steam generation, part load operation and for support during start up or shut down of the reactor system.

The energetic balance according to Example 1 was:

$${t}_{steam} / M {W}_{t h erm . en . gen :} 1.5 - 2.5 {t}_{steam} / M {W}_{t h erm . en . gen}$$

$$Power demand M {W}_{el} / {t}_{steam} : 0.025 - 0.3 M {W}_{el} / {t}_{steam} , preferably 0.075 - 0.25 M {W}_{el} / {t}_{steam}$$

“tₛₜₑₐₘ” means the amount of steam generated in tons, “MW_{therm.en.gen}” means the amount of energy, i.e. the thermal energy generated by the reaction in MW, “MWₑₗ” means the demand of electricity required for the overall reaction in MW.

### Example 2: Hydrogenation of Dinitro Toluene with Heat Exchanger in the Circulation Loop

A hydrogenation of dinitro toluene was carried out as in Comparative Example 1. Contrary to Comparative Example 1, no secondary cooling medium loop was operated. Instead, a set-up as shown in FIG. 2 was used, wherein a heat exchanger, here a steam generator, located in the circulation loop was used to recover (at least partially) the reaction heat, i.e. the thermal energy, which was initially transferred to heat transfer medium in the heat exchanger. The reaction heat of the hydrogenation reaction warmed up the heat exchanger medium (boiling water) so that in the evaporator part of the steam generator, steam was generated, having a pressure in the range of from −0.9 barg to 3 barg, preferably in the range of from −0.7 barg to 1 barg (low pressure steam). The low pressure steam was then, optionally after warm-up in an overheater, subsequently pressurized by mechanical vapor compression to a pressure in the range of from 2 barg to 40 barg, preferably in the range of from 3 barg to 20 barg. A backup cooling system was installed with a separate cooling loop inside of the reactor or a further heat exchanger in the hydrogenation bath circulation loop to remove the reaction heat in case of a shutdown of the steam generation, part load operation and for support during start up or shut down of the reactor system.

The energetic balance according to Example 2 was:

$${t}_{steam} / M {W}_{t h erm . en . gen :} 1.5 - 2.5 {t}_{steam} / M {W}_{t h erm . en . gen}$$

$$Power demand M {W}_{el} / {t}_{steam} : 0.025 - 0.3 M {W}_{el} / {t}_{steam} , preferably 0.075 - 0.25 M {W}_{el} / {t}_{steam}$$

### Example 3: Hydrogenation of Dinitro Toluene with Heat Exchanger in the Primary Cooling Loop

A hydrogenation of dinitro toluene was carried out as in Comparative Example 1. Contrary to Comparative Example 1, no secondary cooling medium loop was operated. Instead, a set-up as shown in FIG. 3 was used, wherein a heat exchanger, here a steam generator, located in the primary cooling loop was used to recover (at least partially) the reaction heat, i.e. the thermal energy, which was initially transferred to the primary cooling medium, from the primary cooling medium. The reaction heat of the hydrogenation reaction warmed up the primary cooling medium. The warm primary cooling medium stream was then used in the heat exchanger, here a steam generator, to generate steam from boiling water, so that in the evaporator part of the steam generator, steam was generated, having a pressure in the range of from −0.9 barg to 3 barg, preferably in the range of from −0.7 barg to 1 barg (low pressure steam). The low pressure steam was then, optionally after warm-up in an overheater, subsequently pressurized by mechanical vapor compression to a pressure in the range of from 2 barg to 40 barg, preferably in the range of from 3 barg to 20 barg. A backup cooling system was installed in the primary cooling loop to remove the reaction heat in case of a shutdown of the steam generation, part load operation and for support during start up or shut down of the reactor system.

### SHORT DESCRIPTION OF THE FIGURES

FIG. 1 shows a setup as used in Example 1. The hydrogenation of dinitro toluene to toluene diamine is performed in a hydrogenation reactor R with an optional stirrer S. The hydrogenation bath mainly consisting of water, solvents, solid catalyst, reactands and products is pumped around through an external loop 1, 2. For removal of the reaction heat, which is released from the reaction within the reactor R, a heat exchanger HE is installed within the reactor R. Heat transfer medium stream HTMS1—here boiling water—coming through feed line 3 to the heat exchanger HE takes up thermal energy in the heat exchanger HE, so that steam is generated, resulting in a gaseous heat transfer medium stream HTMS2 of low pressure, leaves the heat exchanger via line 4. The temperature of HTMS2 is optionally elevated in overheater OH, so that a heat transfer medium stream HTMS2a, which has a higher temperature than HTMS2 but still a low pressure or HTMS2, is transferred to a mechanical compressor MC, where the low pressure heat transfer medium stream HTMS2(a) is compressed, so that a heat transfer medium stream HTMS3(a) having a higher pressure than HTMS2(a) is generated, which leaves the mechanical compressor MC via line 5. For sake of simplicity, only the backup cooling system is shown but not the inlets for feeding liquid heat transfer medium to HTMS2 or HTMS2a before the compressor and not the outlets for taking-off partial heat transfer medium streams HTMS2x or HTMS2ax before the compressor MC.

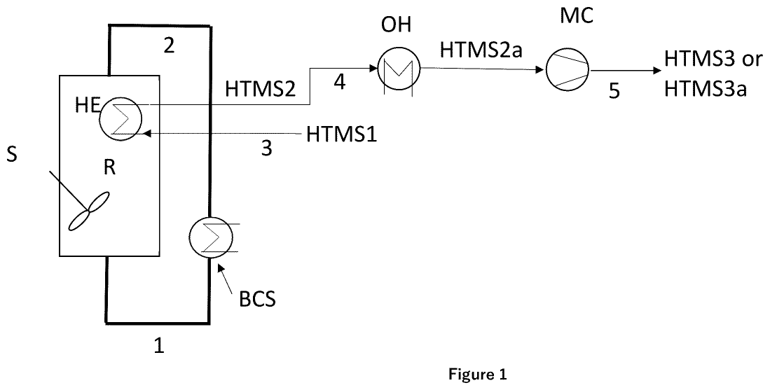

FIG. 2 shows a setup as used in Example 2. The hydrogenation of dinitro toluene to toluene diamine is performed in a hydrogenation reactor R with an optional stirrer S. The hydrogenation bath mainly consisting of water, solvents, solid catalyst, reactands and products is pumped around through an external loop 1, 1a. For removal of the reaction heat, which is released from the reaction within the reactor R, a heat exchanger HE is installed within the circulation loop. Heat transfer medium stream HTMS1—here boiling water—coming through feed line 3 to the heat exchanger HE takes up thermal energy in the heat exchanger HE, so that steam is generated, resulting in a gaseous heat transfer medium stream HTMS2 of low pressure, leaves the heat exchanger via line 4. The temperature of HTMS2 is optionally elevated in overheater OH, so that a heat transfer medium stream HTMS2a, which has a higher temperature than HTMS2 but still a low pressure or HTMS2, is transferred to a mechanical compressor MC, where the low pressure heat transfer medium stream HTMS2(a) is compressed, so that a heat transfer medium stream HTMS3(a) having a higher pressure than HTMS2(a) is generated, which leaves the mechanical compressor MC via line 5. A variant of the backup cooling system is shown with lines 2a, 2b and a separate cooling loop (not shown) inside of the reactor R but, for sake of simplicity, not the inlets for feeding liquid heat transfer medium to HTMS2 or HTMS2a before the compressor and not the outlets for taking-off partial heat transfer medium streams HTMS2x or HTMS2ax before the compressor MC.

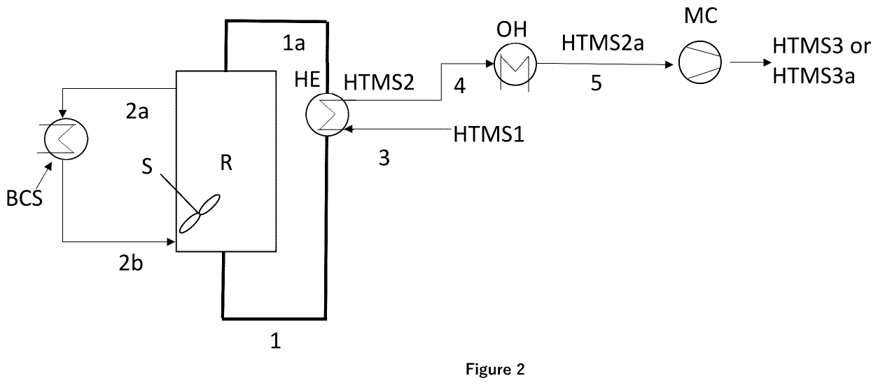

FIG. 3 shows a setup as used in Example 3. The hydrogenation of dinitro toluene to toluene diamine is performed in a hydrogenation reactor R with an optional stirrer S. The hydrogenation bath mainly consisting of water, solvents, solid catalyst, reactands and products is pumped around through an external loop 1. For removal of the reaction heat, which is released from the reaction within the reactor R, a primary cooling medium loop comprising loop lines 2a, 2b and a heat exchanger located in the reactor as an additional heat exchanger HE1 is used. Heat transfer medium stream HTMS1—here boiling water—coming through feed line 3 to the heat exchanger HE takes up thermal energy in the heat exchanger HE, so that steam is generated, resulting in a gaseous heat transfer medium stream HTMS2 of low pressure, which leaves the heat exchanger HE via line 4. The temperature of HTMS2 is optionally elevated in overheater OH, so that a heat transfer medium stream HTMS2a, which has a higher temperature than HTMS2 but still a low pressure or HTMS2, is transferred to a mechanical compressor MC, where the low pressure heat transfer medium stream HTMS2(a) is compressed, so that a heat transfer medium stream HTMS3(a) having a higher pressure than HTMS2(a) is generated, which leaves the mechanical compressor MC via line 5. A variant of the backup cooling system BCS installed in the primary cooling loop downstream of HE is shown but, for sake of simplicity, not the inlets for feeding liquid heat transfer medium to HTMS2 or HTMS2a before the compressor and not the outlets for taking-off partial heat transfer medium streams HTMS2x or HTMS2ax before the compressor MC.

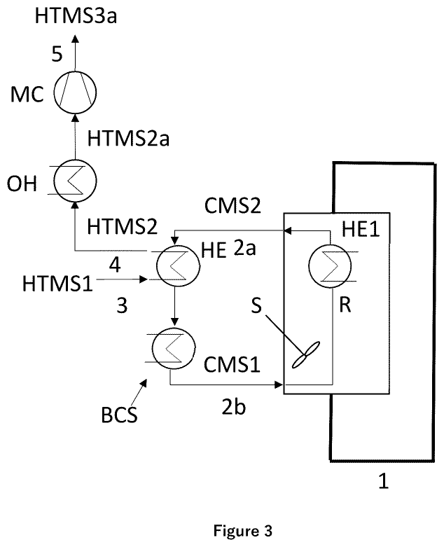

FIG. 4 shows a serial setup for increasing the efficiency by increased temperatures with a primary cooling medium loop and a heat exchanger HE as in FIG. 3. The hydrogenation of dinitro toluene to toluene diamine is performed in a hydrogenation reactor R. The hydrogenation bath mainly consisting of water, solvent, solid catalyst, reactands and products is pumped around through an external loop 1, 1a. For removal of the reaction heat, which is released from the reaction within the reactor R, a primary cooling medium loop comprising loop lines 2a, 2b and 2c and a heat exchanger HE 1 located in the reactor as an additional heat exchanger is used. The warm primary cooling medium stream CMS2 coming from the reactor through loop line 2a comes to an additional heat exchanger HE2, which is installed in the external loop 1, 1a of the hydrogenation bath. The warm primary cooling medium takes up further heat in HE2, whereafter an even warmer primary cooling medium stream CMS4 flows through line 2b in to the heat exchanger HE. Heat transfer medium stream HTMS1—here boiling water—coming through feed line 3 to the heat exchanger HE takes up thermal energy in the heat exchanger HE, so that steam is generated, resulting in a gaseous heat transfer medium stream HTMS2 of low pressure, which leaves the heat exchanger HE via line 4. Due to the heat release in the heat exchanger HE, the primary cooling medium is cooled down and send as cold cooling medium stream CMS3, combined with CMS1 or as CMS1, back through line 2c to the reactor. The temperature of HTMS2 is optionally elevated in overheater OH, so that a heat transfer medium stream HTMS2a, which has a higher temperature than HTMS2 but still a low pressure or HTMS2, is transferred to a mechanical compressor MC, where the low pressure heat transfer medium stream HTMS2(a) is compressed, so that a heat transfer medium stream HTMS3(a) having a higher pressure than HTMS2(a) is generated, which leaves the mechanical compressor MC via line 5. A variant of the backup cooling system BCS installed in the primary cooling loop downstream of HE is shown but, for sake of simplicity, not the inlets for feeding liquid heat transfer medium to HTMS2 or HTMS2a before the compressor and not the outlets for taking-off partial heat transfer medium streams HTMS2x or HTMS2ax before the compressor MC.

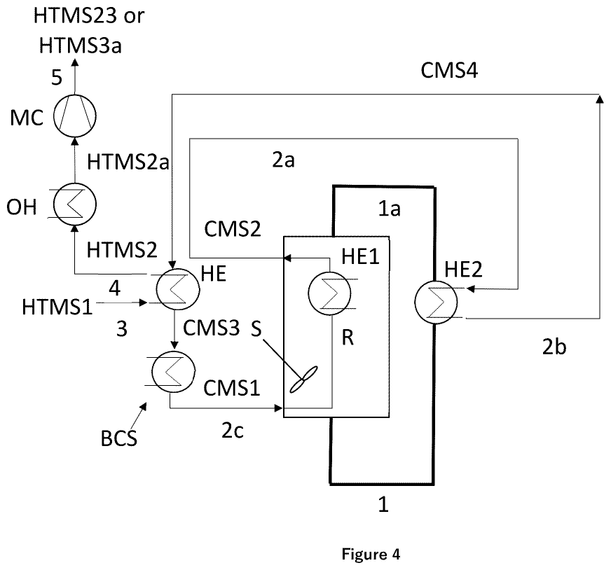

FIG. 5 shows a parallel setup for increasing the efficiency by increased temperatures with a primary cooling medium loop and a heat exchanger HE as in FIG. 3. The hydrogenation of dinitro toluene to toluene diamine is performed in a hydrogenation reactor R. The hydrogenation bath mainly consisting of water, solvent, solid catalyst, reactands and products is pumped around through an external loop 1, 1a. For removal of the reaction heat, which is released from the reaction within the reactor R, a primary cooling medium loop comprising loop lines 2a, 2c and 2d, heat exchanger HE and a heat exchanger HE 1 located in the reactor as an additional heat exchanger is used. Heat transfer medium stream HTMS1—here boiling water—coming through feed line 3 to the heat exchanger H takes up thermal energy in the heat exchanger H, so that steam is generated, resulting in a gaseous heat transfer medium stream HTMS2 of low pressure, which leaves the heat exchanger HE via line 4. Due to the heat release in the heat exchanger HE, the primary cooling medium is cooled down and send a part thereof is sent as cold cooling medium stream CMS3b, combined with CMS1 or as CMS1, back through line 2b to the reactor. A further part of cooling medium stream CMS3 (CMS3a) is sent via line 2c to a heat exchanger HE2, which is installed in the external loop 1, 1a of the hydrogenation bath. The primary cooling medium takes up further heat in HE2, whereafter a primary cooling medium stream CMS4, which has a higher temperature than CMS3(a) flows through line 2d, is combined with the warm primary cooling medium stream CMS2 coming from the reactor through line 2a, and the combined cooling medium stream CMS2+CMS4 flows into the heat exchanger HE. The temperature of HTMS2 is optionally elevated in overheater OH, so that a heat transfer medium stream HTMS2a, which has a higher temperature than HTMS2 but still a low pressure or HTMS2, is transferred to a mechanical compressor MC, where the low pressure heat transfer medium stream HTMS2(a) is compressed, so that a heat transfer medium stream HTMS3(a) having a higher pressure than HTMS2(a) is generated, which leaves the mechanical compressor MC via line 5. A variant of the backup cooling system BCS installed in the primary cooling loop downstream of HE is shown but, for sake of simplicity, not the inlets for feeding liquid heat transfer medium to HTMS2 or HTMS2a before the compressor and not the outlets for taking-off partial heat transfer medium streams HTMS2x or HTMS2ax before the compressor MC.

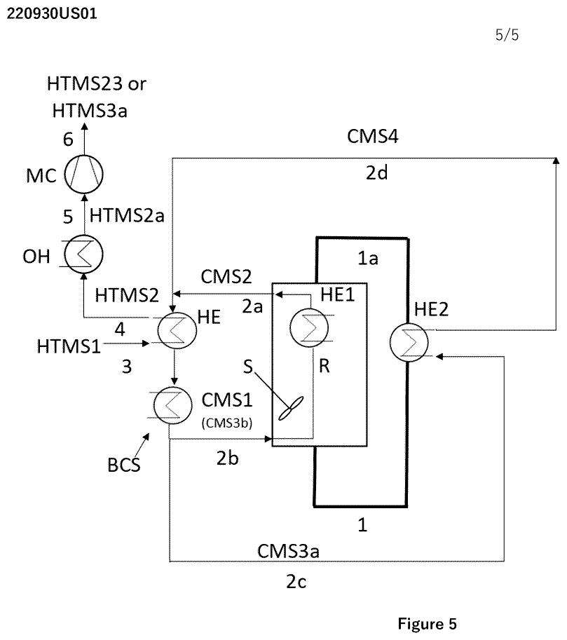

### CITED LITERATURE

WO 00/30743 A1
WO 00/35852 A1
WO 2014/108352 A1

1.-15. (canceled)

16. A hydrogenation process for the conversion of a compound, comprising reacting the compound in a liquid medium with hydrogen in the presence of a heterogeneous hydrogenation catalyst in a reaction vessel comprised in a reaction apparatus, the process further comprising
(i) transferring at least a part of the thermal energy generated in the reaction vessel to a heat transfer medium in a heat exchanger HE, obtaining a heat transfer medium stream HTMS2, which has an increased thermal energy content compared to HTMS1; and
(ii) increasing the pressure of the heat transfer medium stream HTMS2 having a pressure p₂, thereby obtaining a heat transfer medium stream HTMS3, which has an increased pressure p₃ compared to HTMS2.

17. The hydrogenation process of claim 16, being a process for the hydrogenation of a compound having at least one nitro group into the corresponding compound having at least one amino group comprising reacting the compound having at least one nitro group in a liquid medium with hydrogen in the presence of a heterogeneous hydrogenation catalyst in a reaction vessel.

18. The hydrogenation process of claim 16, wherein increasing the pressure of the heat transfer medium stream HTMS2 having a pressure p₂ in (ii) is done by mechanical compression.

19. The hydrogenation process of claim 16, wherein (ii) comprises
(ii.1) increasing the temperature of heat transfer medium stream HTMS2 having a temperature T₂ to a temperature T₂ₐ, which is higher than T₂, thereby obtaining a heat transfer medium stream HTMS2a having a pressure p₂ₐ and a temperature T₂ₐ; and
(ii.2) compressing HTMS2a, thereby obtaining a heat transfer medium stream HTMS3a, which has an increased pressure p₃ₐ compared to HTMS2 and HTMS2a respectively and a temperature T₃ₐ.

20. The hydrogenation process of claim 16, wherein the heat transfer medium comprises water.

21. The hydrogenation process of claim 16, wherein the reaction vessel comprised in the reaction apparatus comprises a reactor selected from the group consisting of (multi)tubular reactor, stirred tank reactor and loop reactor.

22. The hydrogenation process of claim 21, wherein a heat exchanger HE is located within the reaction vessel.

23. The hydrogenation process of claim 21, wherein the reaction apparatus comprises a reaction vessel and a circulation loop, and a heat exchanger HE is located within the circulation loop.

24. The hydrogenation process of claim 21, wherein the reaction apparatus comprises a reaction vessel and a cooling system in connection to the reaction vessel and a heat exchanger HE1 located within the reaction vessel or surrounding the reaction vessel at least partially.

25. The hydrogenation process of claim 24, wherein (i) comprises
(i.1) transferring at least a part of the thermal energy generated in the reaction vessel to a cooling medium in HE 1, obtaining a cooling medium stream CMS2, which has an increased thermal energy content compared to CMS1; and
(i.2) transferring at least a part of the thermal energy comprised in cooling medium stream CMS2 to a heat transfer medium stream HTMS1 in a heat exchanger HE, thereby obtaining a heat transfer medium stream HTMS2, which has an increased thermal energy content compared to HTMS1.

26. The hydrogenation process of claim 23, wherein for the reaction apparatus comprising a reaction vessel and a cooling system in connection to the reaction vessel and a heat exchanger HE1 located within the reaction vessel or surrounding the reaction vessel at least partially, the process comprises:
(i.1.a) transferring at least a part of the thermal energy generated in the reaction vessel to a cooling medium, obtaining a cooling medium stream CMS2, which has an increased thermal energy content compared to CMS1;
(i.1.b) feeding CSM2 to a heat exchanger HE2 installed in the circulation loop of the loop reactor, thereby transferring a further part of the thermal energy generated in the reaction vessel to CMS2 and obtaining a cooling medium stream CMS4, which has an increased thermal energy content compared to CMS2;
(i.2) transferring at least a part of the thermal energy comprised in cooling medium stream CMS4 to a heat transfer medium stream HTMS1 in the heat exchanger HE, thereby obtaining a heat transfer medium stream HTMS2, which has an increased thermal energy content compared to HTMS1; and
(ii) increasing the pressure of the heat transfer medium stream HTMS2 obtained in (i.2) having a pressure p₁, thereby obtaining a heat transfer medium stream HTMS3, which has an increased pressure p₂ compared to HTMS2.

27. The hydrogenation process of claim 23, wherein for the reaction apparatus comprising a reaction vessel and a cooling system in connection to the reaction vessel and a heat exchanger HE1 located within the reaction vessel or surrounding the reaction vessel at least partially, the process comprises:
(i.1.a) transferring at least a part of the thermal energy generated in the reaction vessel to a cooling medium, obtaining a cooling medium stream CMS2, which has an increased thermal energy content compared to CMS1;
(i.1.b) obtaining a cooling medium stream CMS3 from heat exchanger HE, which has a decreased thermal energy content compared to CMS2;
(i.1.c) feeding at least a part of CMS3 (CMS3a) to a heat exchanger HE2 located in the circulation loop of the loop reactor, thereby transferring a further part of the thermal energy generated in the reaction vessel to CMS3a and obtaining a cooling medium stream CMS4, which has an increased thermal energy content compared to CMS3a;
(i.1.d) combining partial cooling medium stream CMS4 with cooling medium stream CMS2 thereby obtaining a combined cooling medium stream CMS2+CMS4;
(i.1.e) feeding the combined cooling medium stream CMS2+CMS4 to the heat exchanger HE;
(i.1.f) transferring at least a part of the thermal energy comprised in combined cooling medium stream CMS2+CMS4 to a heat transfer medium stream HTMS1 in the heat exchanger HE, thereby obtaining a heat transfer medium stream HTMS2, which has an increased thermal energy content compared to HTMS1; and
(ii) increasing the pressure of the heat transfer medium stream HTMS2 obtained in (i.2.a) having a pressure p₁, thereby obtaining a heat transfer medium stream HTMS3, which has an increased pressure p₂ compared to HTMS2.

28. A reaction system for hydrogenation, comprising:
(a) a reactor having
(a.1) means for feeding gaseous and liquid materials into the reactor;
(a.2) means for intermixing in the reactor,
(a.3) an outlet for removing reaction mixture from the reactor;
(a.4) at least one inlet for reintroduction of the reaction mixture;
(a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
(a.6) at least one heat exchanger HE1 arranged within the reactor, outside of the reactor or in the circuit lines (a.5), wherein the heat exchanger HE lhas at least one inlet (a.6.1) for feeding a cooling medium into the heat exchanger and at least one outlet (a.6.2) for removing the cooling medium from the heat exchanger HE1;
(b) a heat exchanger HE comprising
(b.1) at least one inlet (b.1.1) for feeding a cooling medium into the heat exchanger HE, and at least one outlet (b.1.2) for removing the cooling medium from the exchanger HE;
(b.2) at least one inlet (b.2.1) for feeding a heat transfer medium stream into the heat exchanger HE, and at least one outlet (b.2.2) for removing the heat transfer medium stream from the heat exchanger HE;
wherein the heat exchanger HE is built to allow transfer of thermal energy from a cooling medium stream to a heat transfer medium stream;
(c) lines in fluid connection with the at least one heat exchanger HE1 (a.6) and the heat exchanger HE (b), which are built and arranged
(c.1) to allow transfer of the cooling medium stream coming from the at least one outlet of the heat exchanger HE1 (a.6.2) into an inlet of the heat exchanger HE (b.1.1) and reintroduction of the cooling medium stream coming from the at least one outlet (b.1.2) for removing the cooling medium from the heat exchanger HE to the at least one inlet (a.6.1) for feeding a cooling medium into the heat exchanger HE1; and
(c.2) to allow introduction of a heat transfer medium stream into the at least one inlet (b.2.1) of the heat exchanger HE for feeding a heat transfer medium stream into the heat exchanger HE and removal of a heat transfer medium stream from the at least one outlet (b.2.2) of the heat exchanger HE for removing the heat transfer medium stream of the heat exchanger HE;
(d) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.

29. The reaction system of claim 28, wherein the hydrogenation comprises converting a compound having at least one nitro group into the corresponding compound having at least one amino group by hydrogenation.

30. A reaction system for hydrogenation, comprising:
(a) a reactor having
(a.1) means for feeding gaseous and liquid materials into the reactor;
(a.2) means for intermixing in the reactor,
(a.3) an outlet for removing reaction mixture from the reactor;
(a.4) at least one inlet for reintroduction of the reaction mixture;
(a.5) circuit lines outside of the reactor in fluid connection to the outlet (a.3) and inlet (a.4), which enable withdrawal of a reaction mixture from the reactor via outlet (a.3) and reintroduction of the reaction mixture into the reactor via the inlet (a.4);
(b) a heat exchanger HE comprising
(b.1) at least one inlet (b.1.1) for feeding the reaction mixture into the heat exchanger HE, and at least one outlet (b.1.2) for removing the reaction mixture from the exchanger HE;
(b.2) at least one inlet (b.2.1) for feeding a heat transfer medium stream into the heat exchanger HE, and at least one outlet (b.2.2) for removing the heat transfer medium stream from the heat exchanger HE;
wherein the heat exchanger HE is built to allow transfer of thermal energy from a reaction mixture to a heat transfer medium stream; and
(c) means for compression arranged in communication with the at least one outlet (b.2.2) of the heat exchanger HE, built and arranged to allow compression of the heat transfer medium stream coming from the heat exchanger HE from a pressure p₁ to a pressure p₂, wherein p₂ is higher than p₁.

31. The reaction system of claim 30, wherein the hydrogenation comprises converting a compound having at least one nitro group into the corresponding compound having at least one amino group by hydrogenation.

32. The reaction system according to claim 28, further comprising
(x) means for increasing the temperature of the heat transfer medium stream coming from the heat exchanger HE (HTMS2), which has a temperature T₂, to a temperature T₂ₐ, which is higher than T₂, thereby obtaining a heat transfer medium stream (HTMS2a) having a pressure in the range of from p₂−300 mbar to p₂, and a temperature T₂ₐ.
